(19) United States
(12) Patent Application Publication
(10) Pub. No.: US 2004/0209361 A1
(43) Pub. Date: Oct. 21, 2004

(54) UV-cross-linked PVA-based polymer particles for cell culture

(76) Inventors: John J. Hemperly, Apex, NC (US); Chorng-Fure Robin Hwang, Thousand Oaks, CA (US); Steven C. Keith, Chapel Hill, NC (US); Mohammad A. Heidaran, Cary, NC (US)

Correspondence Address: VENABLE LLP, 575 7TH STREET, N.W., WASHINGTON, DC, 20004-1601, US

(21) Appl. No.: 10/418,293

(22) Filed: Apr. 18, 2003

(51) Int. Cl. C12N 5/00

(52) U.S. Cl. 435/404

(57) ABSTRACT

Solid substrates for cell culture are UV-cross-linked PVA-based hydrogel polymer particles. The particles are biocompatible with living cells and support cell adherence. Bioaffecting molecules may be reversibly entrapped within the particles. The particles are capable of forming self-assembled aggregates with cultured cells in aqueous suspension. Preferably, the PVA-based polymer is PVA-SbQ.

### FIELD OF THE INVENTION

The present invention relates to solid substrate particles for cell culture that promote cell adhesion and provide slow release of bioaffecting molecules entrapped within the particles. The present invention also relates to methods for making and using the solid substrate particles. Particularly, the solid substrate particles are useful for anchorage-dependent mammalian cell culture.

### BACKGROUND OF THE INVENTION

Certain cells are anchorage-dependent and require the presence of a surface to which they attach for optimal growth. Attachment permits individual cells to spread out, grow, and organize. A variety of particles have been used as substrates for cell culture. Such particles have been made from calcium compounds, ceramics, glass, cellulose beads, agarose, polystyrene, gelatin, collagen sponge, or other polymers. U.S. Pat. No. 5,006,467 discloses cell culture microcarriers consisting of water insoluble polymer particles of (meth)acrylic ester. U.S. Pat. No. 5,629,191 discloses porous particles having isopycnic density that are formed from a biocompatible matrix by freeze-drying and cross-linking. These particle substrates provide physical support to the cells, but do not allow cell attachment. Lack of cell attachment to the particles results in the cells gradually losing their normal morphology and phenotypic expression, and ultimately limits cell growth and cell differentiation.

To solve this problem, these non-cell-adherent substrates are sometimes covalently modified with cell-adhesion-promoting molecules. For example, Kobayashi et al., Biomaterials, Vol. 12, October, 747-751 (1991), disclose covalently immobilized cell-adhesive proteins on the surface of poly(vinyl alcohol) (PVA) hydrogel by diisocyanates, polyisocyanates, and cyanogen bromide, to promote cell adhesion; Kobayashi et al., Current Eye Research, Vol. 10, No. 10, 899-908 (1991), disclose that cell adhesive proteins and molecules are covalently immobilized onto PVA hydrogel sheets that promote corneal cell adhesion and proliferation. However, covalent modification is not attractive, as it adds steps, chemical cross-linkers, and complexity to the preparation of the particle substrates.

In addition to the requirements for sufficient cell adhesion and cell nutrients, hormones and protein growth factors are essential to cell growth and cell differentiation. Serum contains numerous hormones and protein growth factors, and has been conventionally used to support cell growth in culture. During cell culture, mammalian cells deprived of serum stop growing and become arrested usually between the mitosis and S phase, in a quiescent state called G₀. Although growth factors have been identified and isolated from serum, an effective serum substitute has not been developed. Because serum is expensive and must be replaced every 1-3 days, a suitable serum-free medium or reduced-serum medium is desirable. By “reduced-serum” is meant a solution containing less than 100% serum, preferably less than 50%, more preferably less than 25%, and most preferably less than 15%.

Modification of polymer particles to improve their stability and insolubility in the aqueous solution is common. For example, polylactic acid (PLA), polyglycolic acid (PGA), and polylactic:glycolic acid (PLGA) scaffolds are chemically modified by cross-linking to improve their stability in water. However, the need for a cross-linker and the associated reaction add complexity and possible toxicity to the preparation of particle substrates. Moreover, the properties of particles amenable to cell culture may be adversely affected by the cross-linking reaction.

PVA hydrogel is compatible with cells and has advantages for cell culture, including high water content, softness, bioinertness, and good permeability for cell nutrients including oxygen, glucose, amino acids, lactate, and inorganic ions. However, non-cross-linked PVA hydrogel does not support cell adhesion and allow protein adsorption. See for example, Kawase et al., Biol. Pharm. Bull., 22(9), 999-1001 (1999). Moreover, PVA hydrogel has little or no structural integrity unless firmly attached to a macroscopic support such as a petri dish. Although intramolecular and intermolecular chemical cross-linking improves the stability and insolubility of the PVA hydrogel, it is not desired because it does not support cell adhesion and allow protein adsorption.

### BRIEF SUMMARY OF INVENTION

The present invention provides solid substrate particles for cell culture comprising UV-cross-linked PVA-based hydrogel particles that support cell adhesion. Bioaffecting molecules may be reversibly entrapped within the particles without covalent modification and be slowly released into the culture medium and cells in close proximity. Bioaffecting molecules are those materials required for cell viability and cell growth or that effect cell adhesion to the particle surface. The bioaffecting molecules may be hormones, growth factors, large molecular weight cell nutrients, molecules capable of cell interaction and cell signaling, DNA molecules capable of being taken up by cells, polysaccharides capable of modulating cell adhesion to the polymer coating, or a combination thereof. As a result, the use of reduced amounts of serum or even serum-free culture medium for cell culture becomes feasible.

Preferably, the UV-cross-linked PVA-based hydrogel particles of the present invention are made from a UV-cross-linkable PVA-based polymer such as PVA-(acetalized with N-methyl-4-(p-formyl styryl) pyridinium methosulfate) (PVA-SbQ). The PVA-based polymer particles are cross-linked with UV light, a process that can be easily performed and controlled spatially and temporarily. In addition, UV-cross-linking does not introduce chemical cross-linkers into the substrate.

Preferably, the PVA-based polymer particles produced from the UV-cross-linkable PVA polymer do not readily degrade in liquid culture medium. In one embodiment, the particles may form self-assembled aggregates with the anchorage-dependent cells in liquid cell culture suspension. In another embodiment, the particles may be added to cultured cells that have anchored onto a substrate surface. In some applications, the particles may be embedded into a cell culture substrate to provide controlled release of entrapped bioaffecting molecules.

The present invention also provides methods for making solid substrate particles by spray-drying (SD) or spray-freeze-drying (SFD). The methods involve the steps of atomizing (e.g. spraying) a liquid formulation comprising a UV-cross-linkable PVA-based polymer to form particles, reducing the water content of the particles, and cross-linking the particles with UV light.

The present invention also provides an improvement for cell culture using the solid substrate particles. The particles may be added to cells that have anchored onto a substrate surface; this provides an environment for the controlled release of any entrapped bioaffecting molecules to the cells in an efficient manner. The particles may also serve as a substrate in a liquid suspension cell culture. In this environment, the particles may form self-assembled aggregates with the cultured cells and provide attachment sites as well as various nutrients and growth factors for cell growth. The particles may further be embedded in a solid substrate for cell culture providing for the controlled release of bioaffecting molecules to the cells cultured on the substrate.

The present invention also provides an improvement to a method for treating a subject in need of treatment. The subject is treated with the self-assembled aggregates formed by the solid substrate of the present invention and the cultured cells. The cultured cells may have therapeutic effects and may originate from the subject being treated. The self-assembled aggregates can be injected into the subject through a needle or other cannula. The solid substrate particles used for such treatment may further comprise reversibly entrapped bioaffecting molecules.

### BRIEF DESCRIPTION OF DRAWINGS

FIG. 1 is a schematic drawing showing the self-assembled aggregates comprising cultured cells and UV-cross-linked PVA-based polymer particles of the present invention and the growth factors (GF) entrapped within the particles are control released to the cells.

FIG. 2 is a schematic drawing showing the UV-cross-linked PVA-based polymer particles of the present invention added to cells in culture, wherein the growth factors (GF) entrapped within the PVA-based polymer particles are released to the cells.

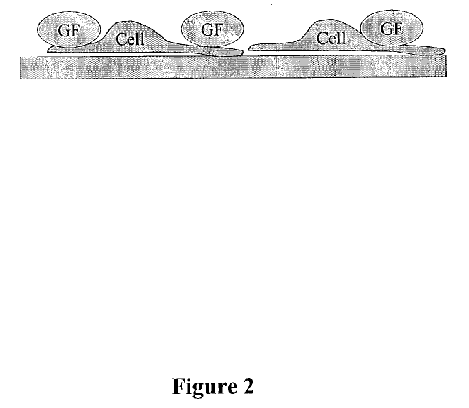

FIG. 3 shows the self-assembled aggregates of cells and the PVA-based particles of the present invention in a liquid suspension cell culture medium.

FIG. 4 is a diagram showing a representative size distribution of the particles of the present invention prepared by either spray-drying (SD) or spray-freeze-drying (SFD).

### DETAILED DESCRIPTION OF INVENTION

The present invention provides UV-cross-linked hydrogel particles made from UV-cross-linkable PVA-based polymer. The PVA-based polymer particles are biocompatible with living cells and insoluble in water. The PVA-based polymer particles of the present invention provide not only physical support but also attachment sites for cells in culture.

The PVA-based polymer particles are hydrogels that swell in water and are particularly compatible with living cells. These particles are capable of entrapping large molecules, so that the diffusional or transportational properties of the entrapped molecules is reduced. Upon exposure to aqueous solution, the hydrogel particles swell to a desired extent, and the transport or diffusional properties of any entrapped large molecules are accordingly altered. The diffusional or transportational properties of the entrapped large molecule are dependent on the size of the molecule and the extent of the cross-linking, and may be controlled by the extent of cross-linking. Optimization of the desired diffusional or transportational properties can be achieved by routine experimentation. The preferred PVA-based polymer particles with entrapped molecules mimic a native extracellular matrix for cells cultured thereon, and provide enhanced cell adhesion even in the absence of adhesion-promoting molecules attached thereto.

Preferably, the PVA-based polymer is PVA-(acetalized with N-methyl-4-(p-formyl styryl) pyridinium methosulfate-) (PVA-SbQ). The amount of SbQ attached to the PVA can vary from about 0.5 mol % to about 10 mol %. Variants of the SbQ moiety exist to provide for use of different wavelength for cross-linking, ranging from about 350 nm to about 600 nm. The more SbQ content in the PVA-based polymer, the faster the UV cure and the greater the cross-linking density of the resultant polymer particles. In general, a sufficiently high degree of cross-linking is desired in the PVA-based polymer particles of the present invention so that the resultant polymer particles are relatively insoluble in water and culture medium. In general, the particles should be sufficiently stable to maintain their integrity over the time that they remain in cell culture.

Suitable PVA-SbQ polymers used in the present invention are preferably free of antimicrobial agents and have neutral pH. (Antimicrobials are added to most PVA-SbQ formulations to improve the shelf life.) An example of suitable PVA-SbQ is the PVA-based polymer designated SPP-LS-400, which is manufactured by Charkit (Darian, Conn.). In this particular PVA-SbQ sample, no antimicrobial agents are present. The characteristics of this PVA-SbQ polymer are: degree of polymerization (DP), 500; degree of saponification (DS), 88%; SbQ content in molar percentage, 4.1±0.15; solid content, 13.3%; pH of the polymer, 5.5 to 7; and viscosity at 25° C., 2000±500 cp.

The PVA-based polymer particles are cross-linked with UV light. UV-cross-linking provides a simple process for stabilization of the polymer and further entrapment of the bioaffecting molecules. UV-cross-linking avoids the use of other chemicals such as cross-linker and reaction initiators, and thus avoids the problems of introducing potentially toxic materials to cultured cells or changing the properties of the polymer material. In addition, there is less concern for potential side reactions caused by chemical cross-linking. The UV-cross-linking reaction is a simple process that can be controlled both spatially and temporally, as one may selectively cross-link the particles by limiting the amount of UV irradiation to certain areas for selected time periods.

The PVA-based polymer particles of the present invention are preferably uniform in shape, most preferably approximately spherical with a diameter of less than 35 microns. The particles preferably have a narrow size distribution. These characteristics may be varied by the method used to make the particles, as will be appreciated by persons of skill in the art.

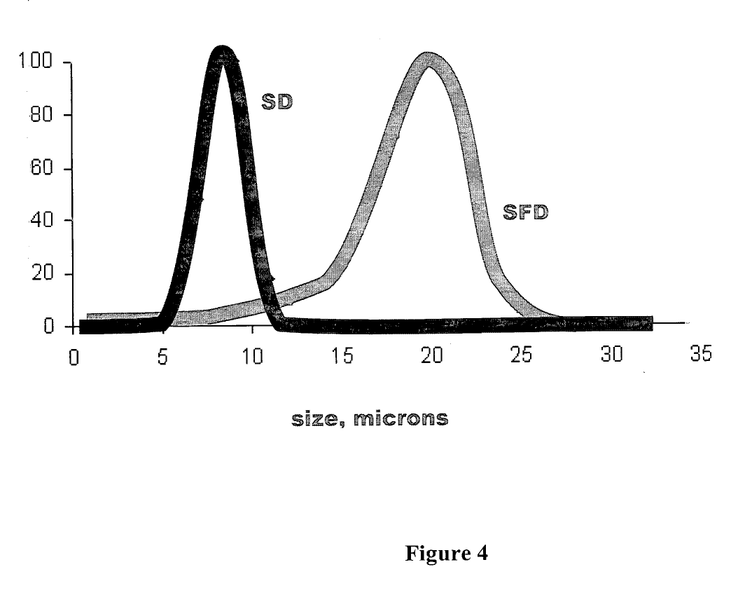

As indicated in FIG. 4, when the particles are prepared by spray-drying (SD), the majority of the particles are within the range of about 5-13 microns, with about 9 microns being the peak of density distribution, and all particles are less than about 13 microns. When the particles are prepared by spray-freeze-dry (SFD), the majority of the particles are within the range of about 1-28 microns with about 20 microns being the peak of the density distribution, and all particles are less than about 28 microns. All the particles prepared by either SD or SFD have a size of less than 35 microns in diameter. Conditions for SD or SFD are set forth below.

The UV-cross-linked PVA-based polymer particles of the present invention provide enhanced cell adhesion even in the absence of adhesion-promoting molecules attached to the particles. Unlike the UV-cross-linked PVA-based polymer particles of the present invention, other particles must be covalently attached to adhesion-promoting molecules in order to support cell adhesion. Enhanced cell adhesion on the UV-cross-linked PVA-based polymer particles may be associated with the multiplicity of hydroxyl groups or the result of cross-linking of these hydroxyl groups.

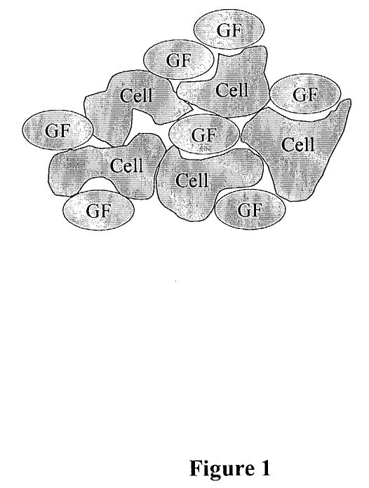

The self-assembly property of the PVA-based particles of the present invention refers to the ability of the particles, upon addition to aqueous cell culture suspension, to form intricate assemblies with the cultured cells in the liquid suspension and yield parallel three-dimensional aggregates. The particles are mixed with a suspension of living cells at a concentration such that the cells form the majority of the resulted aggregate, as shown schematically in FIG. 1. In the resulting environment, most cultured cells are in the assembled aggregates, few cells are not within the aggregates. The self-assembled aggregates of the particles and culture cells are especially observable in cell culture vessels with inner surfaces that do not support cell adhesion.

The polymer particles may also contain entrapped bioaffecting molecules, which are molecules required for cell viability, cell growth, cell differentiation, or affecting cell adhesion to the culture surface. These bioaffecting molecules may be hormones, growth factors, large molecular weight cell nutrients, molecules capable of cell interaction and cell signaling, DNA molecules capable of being taken up by cells, polysaccharides capable of modulating cell adhesion to the polymer coating, or a combination thereof. Possible growth factors include epidermal growth factor, fibroblast growth factor, platelet-derived growth factor, nerve growth factor, transforming growth factor-β, hematopoietic growth factors, interleukins, or any combination thereof. Large molecular weight cell nutrients may include, for example, protein nutrients that are beneficial for certain types of mammalian cell culture. Polysaccharides capable of modulating cell adhesion to the PVA-based polymer include, for example, hyaluronic acid. Further, the particles may comprise a growth-promoting peptide chemically coupled to the PVA backbone. One preferred peptide for this purpose is arginine-glycine-aspartic acid (RGD).

Upon exposure to aqueous solution, the hydrogel particles of the present invention swell, and large molecules entrapped within the particles are transported inside the hydrogel network and presented to cells that are attached to the particles. Smaller molecules such as cell nutrients that are not entrapped within the particles but present in the culture medium are also easily transported into and out of the hydrogel particles and presented to the cells attached thereto. The effective transport of the cell nutrients is particularly beneficial to cells located in the core of the self-assembled aggregates. This provides sufficient nutrients for the cells growing inside the aggregates. Otherwise, these cells are not exposed to the outside cell culture medium and nutrients, and when they take up all available cell nutrients, they become deprived of nutrients and form a necrotic center that is harmful for overall cell growth. The hydrogel particles of the present invention provide not only support for cell adhesion but also an efficient presentation of the biologically active molecules to the attached cells, as the particles are in contact with or close proximity to the cultured cells.

In one preferred embodiment, bioaffecting molecules are reversibly entrapped in the hydrogel particles and slowly released into the culture medium. Cells respond well to the relatively low levels of the bioaffecting molecules entrapped within the particles. The bioaffecting molecules exert their effects on cells cultured on the particles and the effects are maintained over 3-14 days. Most preferably they are maintained over 3-7 days. As a result, less serum-containing medium and/or growth factors and other medium additives are required for the cell culture. Since the bioaffecting molecules are released over time, the culture medium need not be replaced as frequently as in normal cell culture. The release rate of the entrapped molecule is controlled by a variety of factors, including the load amount of the bioaffecting molecules, the size of the entrapped molecules, the degree of cross-linking, the porosity of the particles, the size and size distribution of the particles, the particle shape, and the molecular weight or type of PVA.

It is speculated that the slow release and controlled release offered by the PVA-based particles of the present invention is probably due to their polymeric structure. Long-chain polymers in their native state generally offer a diffusion barrier to molecules, as the chains are closely spaced and form voids in between. When the polymers are cross-linked, the spacing between the polymer chains is further fixed and the chains are less capable of moving aside for diffusing molecules. As a result, the polymer chains are more resistant to movement, and the diffusion barrier to entrapped molecules increases. Therefore, compared with non-cross-linked particles, UV-cross-linked PVA-SbQ particles of the present invention give the polymer a more rigid backbone, making the polymer relatively stable in solution and providing a larger diffusion barrier to the entrapped molecule.

The diffusion rate of the entrapped bioaffecting molecules is preferably controlled by varying the UV-cross-linking density. The UV-cross-linking density is adjusted by the content of the SbQ moiety in the PVA-based polymer or the conditions for cross-linking such as time and wavelength. By selecting the proper weight percentage of the solution and the molar percentage of the SbQ moiety in the PVA-based polymer, one can change the cross-linking density of the particles, and thus, alter and tailor the diffusion and control release properties of the entrapped molecules for needs of slow, sustained, and/or controlled release.

The self-assembled aggregates of cells and the particles are also suitable for implantation or injection through syringe needle or cannula. Injection or implantation of the aggregates avoids damage to the cells, as the cells need not be trypsinized or otherwise chemically or mechanically treated for release from a culture device. This is particularly useful for cells such as neurons or neuronal cells differentiated from stem cells or progenitor precursors in vitro, as these cells have long and intertwined morphology.

The PVA-based particles of the invention are especially useful for tissue engineering. Preferably, the cells in the self-aggregates reorganize and form functional networks that mimic the natural tissue. When cells taken from patients are cultured ex vivo in the self-aggregates, synthetic tissue becomes feasible.

The PVA-based particles of the invention may also be added to cells that have already anchored on the surface of a cell culture device. Instead of forming self-assembled aggregates with the cultured cells, the particles settle onto the cells and attach to the anchored cells to provide slow release or controlled release of the bioaffecting molecules reversibly entrapped within the particles.

The PVA-based particles of the invention may also be embedded into a polymer substrate for cell culture. The UV-cross-linked particles may be added to and mixed with the polymer solution that ultimately forms the polymer substrate for cell culture; the UV-cross-linked particles may also be mechanically implanted or seeded inside a substrate for cell culture. The UV-cross-linked particles are suitable for use in all liquid cell culture media, in which it is desired that the particles exert slow release of the entrapped molecules. Preferably, the polymer substrate is a hydrogel that swells in water and that may be modified covalently or non-covalently with cell-adhesion promoting molecules. The embedded particles provide slow and controlled release of the bioaffecting molecules entrapped therein to the cells cultured on the polymer substrate.

The particles of the present invention may preferably be prepared by spray-drying or spray-freeze-drying process as follows:

1. Preparing the Liquid Formulation.

The native PVA-based polymer, such as the suitable PVA-SbQ disclosed above, is diluted and dissolved in water to make a liquid formulation (i.e. solution), with the degree of dilution depending on the desired properties of the hydrogel particles. One of ordinary skill in the art will be able to make particles from formulations with a wide range of DP, DS, SbQ content, solids content, and final dilution.

The PVA-SbQ polymer, which is commercially available as a 13% solution (w/v (13 grams of PVA-SbQ dissolved in 100 ml solution)) is diluted in water to obtain a PVA-SbQ polymer solution of about 1-13% (w/v). Preferably, the PVA-SbQ polymer solution has a concentration of about 1-7% (w/v). More preferably, the PVA-SbQ polymer solution has a concentration of about 1.3-5% (w/v).

Bioaffecting molecules may also be added to the solution and dispersed or dissolved in the solution with the PVA-based polymer. These molecules are preferably large enough (greater than or equal to about 5,000 Daltons) so that they may be physically entrapped within the PVA-based polymer particles after UV-cross-linking. There is no specific requirement for the conditions in which the bioaffecting molecules are dissolved as long as they remain stable in solution. Suspensions, emulsions, nanoparticles, microparticles, and the like, of bioaffecting molecules in combination with the UV-cross-linkable PVA are within the scope of the instant invention. The final concentration of the bioaffecting molecules in the UV-cross-linked particles can range from the minimum required to exert a biological effect on cultured cells to the solubility limit in the solution containing the PVA-based polymer. Even highly concentrated bioaffecting molecules that form insoluble aggregates in solution may be useful if the aggregate dissolves over time in the presence of liquid cell culture medium to release the bioaffecting molecules. Preferably, the concentration of the bioaffecting molecule is about 0.01 ng/ml to 3000 ng/ml.

2. Creating the Liquid Formulation

The solution containing the PVA-SbQ polymer and optionally, the bioaffecting molecules (i.e., liquid formulation), is atomized or sprayed to form particles of controlled sizes and size distribution. Spray-drying, spray-freeze-drying, and related methods can be used for the formation of the particles.

The process of spray-drying is commonly used for particle formation. Spray-drying involves transforming a fluid, pump-able medium into a dry-powdered or particle form. The PVA-based particles of the present invention may also be formed by spray-drying the liquid PVA formulation. This is achieved by atomizing the fluid into a drying/heating chamber, where the liquid droplets are passed through a hot-air stream. The objective is to produce a spray of high surface-to-mass ratio droplets (ideally of equal size), then to uniformly and quickly evaporate the water. Evaporation keeps product temperature to a minimum, so little high-temperature deterioration occurs.

The process generally involves the atomization of a liquid feedstock into a spray of droplets and contacting the droplets with hot air in a drying chamber to remove the moisture in the droplets. The sprays are produced by either rotary (wheel) or nozzle atomizers. The feed can be a solution, a suspension or a paste in the simplest form. Evaporation of moisture from the droplets and formation of dry particles proceed under controlled temperature and airflow conditions. (In some instances, this is under a vacuum.) The dried product, which can be varied depending on the feed, dryer design and process conditions, is discharged continuously from the drying chamber. Operating conditions and dryer design are selected according to the drying characteristics of the dried product and specification. The dried product can be powdered, granulated or agglomerated. Though quite energy-intensive in many cases, spray-drying is often the drying method of choice if thermal stability of the product is not an issue. It delivers a powder of specific particle size and moisture content regardless of the dryer capacity or product heat sensitivity. Detailed description of various suitable means of forming dried particles can be found in U.S. patent application Ser. No. 10/299,012, filed Nov. 19, 2002, which is incorporated herein by reference.

The process of spray-freeze-drying, in which a solution is atomized, frozen rapidly, and dehydrated by sublimation, is used for making fine powders for various uses. In one embodiment of the process, a liquid feed containing a dissolved solid is first atomized to form small droplets that are rapidly frozen in a cold air stream. The air may then also be used to dry the frozen particles by sublimation. This is possible if the partial pressure of water vapor (not necessarily the total pressure) is below the saturation vapor pressure of water at that temperature. Air at atmospheric pressure can thus be used, if it is of sufficiently low humidity. The powder particles produced by the process have controlled particle size and spherical morphology. Using this process, one can prepare dry powder particles loaded with bioaffecting molecules such as growth factors, hormones, large molecular weight cell nutrients, molecules capable of cell interaction and cell signaling, DNA molecules capable of being taken up by cells, polysaccharides capable of modulating cell adhesion to the polymer coating, or a combination thereof. The process has been shown to have a benign effect on biological molecules. See, for example, Rogers et al., Drug Development and Industrial Pharmacy, 27(10), 1003-1015 (2001), Giunchedi et al., STP Pharma. Sciences, 5(4), 276-290 (1995), and Risch, Encapsulation and Controlled Release of Food Ingredients, 590, 2-7 (1995).

Liquid formulations of the invention can be atomized by any of a variety of conventional procedures. For example, the liquid can be sprayed through a two-fluid nozzle, a pressure nozzle, or a spinning disc, or atomized with an ultrasonic nebulizer or a vibrating orifice aerosol generator (VOAG). In one embodiment, a liquid formulation is atomized with a pressure nozzle such as a BD AccuSpray™ nozzle.

Atomization conditions may be optimized such that the mean mass diameter of the atomized droplets (e.g., nebulized droplets) is within a desired range. Methods to optimize the generation of droplets of the desired size are conventional. Among the conditions that can be varied to control atomization are gas flow, gas pressure, liquid flow rate, and the type and size of the nozzle can be varied.

Liquid drop size can be readily measured, using conventional techniques, such as laser diffraction. The size of dried particles can be measured by conventional techniques, such as, scanning electron microscopy (SEM).

Following the atomization of a liquid formulation, the droplets may be rapidly frozen to form solid particles. In such instances, the droplets are preferably frozen immediately, or substantially immediately, after the atomization step by passing through a cold fluid (liquid or gas).

In one embodiment, the droplets are frozen by immersing them in a cold liquid that is below the freezing point of the liquid formulation from which the atomized droplets were formed. In a preferred embodiment, the temperature of the cold liquid is about −200° C. to −80° C., more preferably between about −200° C. to −100° C., most preferably about −200° C. (liquid nitrogen is about −196° C.). Any suitable cold liquid may be used, including liquid nitrogen, argon and hydrofluoroethers, or a compressed liquid, such as compressed fluid CO₂, helium, propane or ethane, or equivalent inert liquids, as is well known in the art. For example, in one embodiment, a liquid preparation is atomized through a spray nozzle that is positioned above a vessel containing a suitable cold liquid, such as, liquid nitrogen. The droplets freeze instantaneously upon contact with the cold liquid.

In another embodiment, the droplets are frozen by passage through a gas (e.g., cold air, nitrogen, helium or argon), in a cooling chamber, wherein the gas is below the freezing point of the droplets. In a preferred embodiment, the cold gas is about −5° C. to −60° C., more preferably between about −20° C. to −40° C. The gas can be cooled by conventional methods, such as by cooling coils, heat exchangers or chiller condensers. The temperature of the gas can also be reduced with conventional procedures, e.g., with liquid nitrogen, solid carbon dioxide or an equivalent cryogenic agent to produce the subfreezing temperatures.

Following the formation of solid frozen particles, the particles are dried to produce a powder. By “dry” is meant having a negligible amount of liquid, e.g., having a moisture content such that the particles are readily dispersible to form an aerosol. This moisture content is generally below about 15% by weight water, with less than about 10% being preferred and less than about 1% to about 5% being particularly preferred.

In one embodiment of the invention, the spray-freeze drying is accomplished by lyophilization (freeze-drying, under vacuum), using a conventional lyophilization apparatus. For example, in one embodiment, when particles have been frozen by spraying them into a vessel (such as a Virtis freeze-drying flask) containing liquid nitrogen, the vessel can then be attached to a conventional lyophilizer and the excess liquid nitrogen evaporated off. The frozen aerosol is typically dried within about 48 hours and reaches a moisture level below about 1 wt %. Alternatively, droplets that have been frozen in cold air at about atmospheric pressure and, optionally, partially dried at about atmospheric pressure (as is discussed below) can then be placed in a lyophilization flask and subjected to lyophilization.

In another embodiment, the frozen droplets are dried by sublimation in a cold, desiccating gas (e.g., air, nitrogen or helium) stream at about atmospheric pressure. By “about atmospheric pressure” is meant herein as a pressure ranging from about one half atmosphere to about five atmospheres.

The temperature of the gas can be reduced by any of a variety of conventional procedures, e.g., with liquid nitrogen, solid carbon dioxide or an equivalent cryogenic agent. Particles of the invention that are dried in such a manner are sometimes referred to herein as “spray freeze atmosphere dried” particles. In a preferred embodiment, atomized droplets are frozen and dried in the same “spray freeze atmosphere dry” chamber, allowing the freezing and drying procedures to be carried out in a single step.

One apparatus and method for drying solid, frozen particles in cold air at about atmospheric pressure is disclosed in Leuenberger, U.S. Pat. No. 4,608,764. Other types of conventional apparatus can also be used.

In a preferred embodiment, frozen atomized particles are dried in a cold gas at about atmospheric pressure in the presence of conditions that enhance fluidization of the particles. Such conditions are known to those of skill in the art and include, e.g., vibration, internals, mechanical stirring, acoustic/sound wave vibration, or combinations thereof, during the drying process. In a particularly preferred embodiment, the frozen, atomized particles are dried in the presence of vibration, internals, mechanical stirring, or combinations thereof, during the drying process. The term, “internals,” as used herein, refers to any physical barrier inside a chamber (e.g., the SFD chamber) or fluidized bed, such as, e.g., blades, plates or other barriers. Such treatments allow the particles to achieve a fluidized state.

In another embodiment, the frozen droplets are dried by a combination of sublimation in a cold, desiccated gas (e.g., air) stream at about atmospheric pressure, as described above, and lyophilization. For example, a composition that has been partially dried at about atmospheric pressure (e.g., to form a cake or a powder that still contains undesirable amounts of liquid) is removed to a lyophilizer, in which the composition is dried further.

Conventional methods can be used to collect the dried compositions. In one embodiment, the dried particles are collected on a filter, from which they can be removed for use. In another embodiment, the spray freeze atmosphere dried particles are collected in a product vessel. Partially dried particles may form a loose cake, from which remaining moisture can be removed by further atmospheric sublimation in a cold desiccated air stream, or they can optionally be removed to a lyophilizer or other suitable device and further dried under reduced pressure (below atmospheric pressure.)

Particles dried by any of the above methods exhibit substantially the same properties (e.g., particle size, porosity, and the like).

The atmospheric spray freeze drying process, especially with vibration and/or internals, provides an economically feasible method of producing dried particles and increasing yield. Unlike the spray-freeze-drying process disclosed in, e.g., U.S. Pat. No. 6,284,282 to Maa, this embodiment of the invention produces dried particles with a single apparatus (in single step). Other spray-freeze-dried processes utilized for preparing pharmaceutical compositions often include a second step of lyophilization, which involves removing the frozen particles from the spray-freezing chamber and transferring the particles to a lyophilizer. This additional step reduces the production feasibility of the spray freeze dry process and can result in agglomeration of the particles due to the moisture still entrapped in the particles, but may be suitably used in the present invention in certain circumstances.

3. Cross-Linking the Particles with UV Light.

The PVA-based polymer particles are treated with UV light to accomplish the cross-linking reaction. The SbQ moiety may preferably be selected to cross-link at a particular wavelength of light, preferably such wavelength is that which minimizes photo-induced damage to the entrapped bioaffecting molecule or provides manufacturability benefit. One of ordinary skill in the art will be able to select the appropriate SbQ moiety, wavelength of light, and time for UV irradiation depending on the desired properties of the particles such as sizes and size distribution and the desired degree of completeness of cross-linking. Variants of the SbQ moiety exist to provide for use of different wavelength radiation for cross-linking, ranging from about 350 nm to about 600 nm. The UV cross-linking reaction typically takes from about 5 seconds to 20 minutes, and preferably, about 10 seconds to 10 minutes.

The following examples are illustrative, but do not limit the scope of the present invention. Reasonable variations, such as those that occur to the reasonable artisan, can be made herein without departing from the scope of the present invention.

### EXAMPLE 1

### Making the PVA-Based Polymer Particles of the Present Invention

Particles with no entrapped bioaffecting molecules were prepared as follows: Solutions containing PVA-SbQ polymer were prepared at a concentration of 1.3% and 5% (w/v). The solutions were processed by spray-drying or spray-freeze-drying to form particle powders, respectively. The particles were cured under a 450 W UV light for about 10 minutes, with manual mixing about every minute.

Particles containing insulin and platelet-derived growth factors were also prepared. Fifty milliliters of PVA-SbQ solution at a concentration of 1.3% or 5% (w/v) was mixed with a 60 μl insulin solution having a concentration of 50 μg/μl and 75 μl platelet-derived growth factor-A solution having a concentration of 0.2/g/ml. The mixture was then spray-dried or spray-freeze-dried to form particles and cured under a 450 W UV light for about 10 minutes, with manual mixing about every minute.

### EXAMPLE 2

### Cell Culture on the PVA-Based Polymer Particles of the Present Invention

The spray-dried or spray-freeze-dried particles as made in Example 1 were added to 75 μl BITS medium, and a series of the BITS media containing different concentrations of the particles were obtained. MC3T3-E1 osteoblast cell suspension was added to the BITS medium containing the UV-cross-linked PVA-based polymer particles to obtain a concentration of approximately 3×10⁶ cells/ml. The resulting cell culture suspensions in BITS media containing various concentrations of the UV-cross-linked PVA-based polymer particles were placed on a rotating plate and cultured in an incubator at 37° C. overnight. The cells were then stained with DAPI (to reveal cell nuclei) for observation.

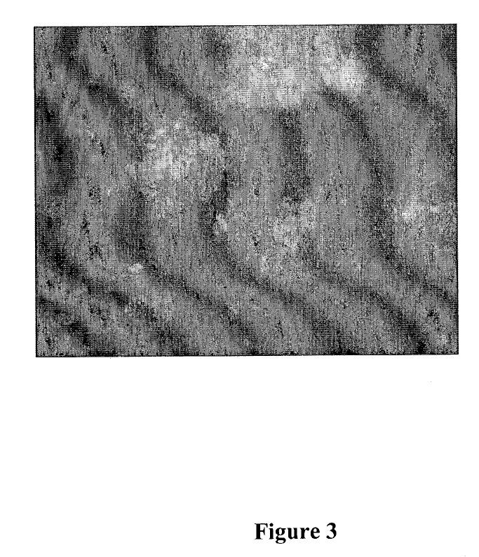

Cell adhesion and cell growth were observed in the liquid cell suspension. Cells and the UV-cross-linked PVA-based polymer particles formed self-assembled aggregates in the liquid suspension with few cells outside the aggregates. In most cell cultures, cells were embedded in the PVA particles as a “mat” that adhered to the plastic bottom of the culture plate. FIG. 3 shows a cell culture containing floating aggregates of cells embedded in the PVA-SbQ polymer particles that would be useful for growth of adherent cells as suspended particle aggregates for in vivo implantation.

We claim:

1. A solid substrate for cell culture comprising
hydrogel particles of a UV-cross-linkable polyvinyl alcohol (PVA)-based polymer,
wherein said hydrogel particles have been cross-linked with UV light to form UV-cross-linked PVA-based polymer particles.

2. The solid substrate of claim 1 having the property of cell adherence.

3. The solid substrate of claim 1, wherein the PVA-based polymer is PVA-(acetalized with N-methyl-4-(p-formyl styryl) pyridinium methosulfate) (PVA-SbQ).

4. The solid substrate of claim 3, wherein the SbQ moiety in the PVA-SbQ is 0.5 to 10 mol %.

5. The solid substrate of claim 3, wherein the PVA-SbQ is free of antimicrobial agents.

6. The solid substrate of claim 1, wherein the PVA-based polymer particles are biocompatible.

7. The solid substrate of claim 6, wherein the PVA-based polymer particles are non-biodegradable.

8. The solid substrate of claim 1, wherein the PVA-based polymer particles are capable of forming self-assembled aggregates with anchorage-dependent cells in liquid suspension cell culture.

9. The solid substrate of claim 1, wherein the PVA-based polymer particles are approximately spherical.

10. The solid substrate of claim 1, wherein the PVA-based polymer particles have a size of less than or equal to about 35 microns in diameter.

11. The solid substrate of claim 10, wherein the PVA-based polymer particles are prepared by spray-drying and have a size of less or equal to about 13 microns in diameter.

12. The solid substrate of claim 10, wherein the PVA-based polymer particles are prepared by spray-freeze-drying and have a size of less than or equal to about 28 microns in diameter.

13. The solid substrate of claim 1, further comprising one or more bioaffecting molecules reversibly entrapped within the PVA-based polymer particles.

14. The solid substrate of claim 13, wherein concentration of the bioaffecting molecule(s) in the PVA-based polymer particles is about 0.01 ng/ml to 3000 ng/ml.

15. The solid substrate of claim 13, wherein the bioaffecting molecules are selected from the group consisting of growth factors, hormones, large molecular weight cell nutrients, molecules capable of cell interaction and cell signaling, DNA molecules capable of being taken up by cells, polysaccharides capable of modulating cell adhesion to the polymer particles, and combinations thereof.

16. The solid substrate of claim 15, having at least one growth factor selected from the group consisting of epidermal growth factor, fibroblast growth factor, platelet-derived growth factor, nerve growth factor, transforming growth factor-β, hematopoietic growth factors, interleukins, and combination thereof.

17. The solid substrate of claim 13, wherein the bioaffecting molecule is hyaluronic acid.

18. The solid substrate of claim 13, wherein the bioaffecting molecules that are reversibly entrapped within the PVA-based polymer particles are released therefrom over time when placed in a cell culture.

19. The solid substrate of claim 1, further comprising a growth-promoting peptide chemically coupled to a PVA backbone of the PVA-based polymer.

20. The solid substrate of claim 19, wherein the growth-promoting peptide is arginine-glycine-aspartic acid (RGD).

21. A method for making a solid substrate for cell culture, comprising
preparing a liquid formulation of UV-Cross-linkable polyvinyl alcohol (PVA)-based polymer,
atomizing the liquid formulation of UV cross-linkable polyvinyl alcohol (PVA)-based polymer to produce an atomized formulation,
drying the atomized formulation at atmospheric pressure to produce dried PVA-based polymer particles, and
cross-linking the dried PVA-based polymer particles with UV light.

22. The method of claim 21, wherein the liquid formulation has a UV-cross-linkable PVA-based polymer concentration of 1-13% (w/v).

23. The method of claim 22, wherein the liquid formulation has a UV-cross-linkable PVA-based polymer concentration of 1-7% (w/v).

24. The method of claim 23, wherein the liquid formulation has a UV-cross-linkable PVA-based polymer concentration of 1.3-5% (w/v).

25. The method of claim 21, wherein the UV-cross-linkable PVA-based polymer is PVA-SbQ.

26. The method of claim 21, wherein the liquid formulation is atomized and dried by spraying into a heated chamber.

27. The method of claim 21, wherein the liquid formulation is atomized by spraying into a cold fluid wherein the PVA particles are frozen.

28. The method of claim 27, wherein the PVA-based polymer particles are dried by lyophilization.

29. The method of claim 21, wherein the liquid formulation further comprises bioaffecting molecules reversibly trapped therein.

30. The method of claim 29, wherein concentration of the bioaffecting molecules in the formulation is about 0.01 ng/ml to 3000 ng/ml.

31. A method for improved cell culture comprising culturing cells in presence of the solid substrate of claim 1.

32. The method of claim 31, wherein the PVA-based polymer particles further comprise bioaffecting molecules reversibly entrapped therein.

33. The method of claim 32, wherein the cells are cultured in cell culture medium containing reduced serum.

34. The method of claim 32, wherein the cells are cultured in serum-free cell culture medium.

35. The method of claim 32, wherein the UV-cross-linked PVA-based polymer particles are added to cells that have anchored in cell culture and provide slow release of the entrapped bioaffecting molecules to the cells in contact.

36. The method of claim 31, wherein the cells are cultured in liquid suspension.

37. The method of claim 31, wherein the cells are cultured in a layer on a substrate surface.

38. The method of claim 31, wherein the UV-cross-linked PVA-based polymer particles are added to liquid cell culture suspension in which the PVA-based polymer particles form self-assembled aggregates with cells cultured therein.

39. The method of claim 31, wherein the UV-cross-linked PVA-based polymer particles are embedded within a polymer substrate for cell culture and the polymer substrate is a hydrogel polymer.

40. The method of claim 39, wherein the UV-cross-linked PVA-based polymer particles comprise bioaffecting molecules.

41. In a method for treating a subject, the improvement comprising treating the subject with self-assembled aggregates formed by the solid substrate of claim 1 and cultured cells.

42. The method of claim 41, wherein the cultured cells have therapeutic effects.

43. The method of claim 41, wherein the cultured cells originate from the subject being treated.

44. The method of claim 41, wherein the self-assembled aggregates are injected into the subject through a needle or cannula.

45. The method of claim 41, wherein the PVA-based polymer particles further comprise reversibly entrapped bioaffecting molecules.
